(19) United States
(12) Patent Application Publication
(10) Pub. No.: US 2003/0045734 A1
(43) Pub. Date: Mar. 6, 2003

(54) Process for the preparation of alkene oxides from alkenes

(76) Inventors: Markus Weisbeck, Koln (DE); Christoph Zurlo, Leverkusen (DE); Gerhard Wegener, Mettmann (DE); Gunter Schummer, Pulheim (DE); Georg Wiessmeier, Gladbach (DE)

Correspondence Address: BAYER CORPORATION, PATENT DEPARTMENT, 100 BAYER ROAD, PITTSBURGH, PA, 15205, US

(21) Appl. No.: 10/210,693

(22) Filed: Jul. 31, 2002

(30) Foreign Application Priority Data
Aug. 2, 2001 (DE) 10137784.3

(51) Int. Cl. C07D 301/06; C07C 27/00

(52) U.S. Cl. 549/532; 568/959

(57) ABSTRACT

The present invention relates to a process for the catalytic partial oxidation of hydrocarbons. This process comprises passing a reaction mixture comprising hydrocarbons, oxygen, and at least one reducing agent, through a catalyst-containing layer to partially oxidize the hydrocarbon, and adsorbing the partially oxidized hydrocarbon in a downstream adsorbent-containing layer.

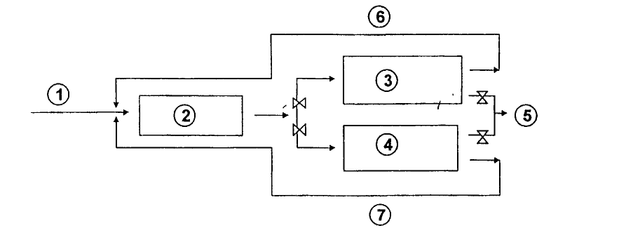

### BACKGROUND OF THE INVENTION

The present invention relates to a process for the catalytic partial oxidation of hydrocarbons in the presence of oxygen and of at least one reducing agent. This process comprises passing the reaction mixture through a catalyst-containing layer, and adsorbing the partially oxidized hydrocarbon in a downstream adsorbent-containing layer.

The catalytic gas-phase partial oxidation of hydrocarbons in the presence of molecular oxygen and a reducing agent is known and described in, for example, DE-A1-199 59 525, DE-A1-100 23 717, U.S. Pat. No. 5,623,090, WO-98/00413-A1, WO-98/00415-A1, WO-98/00414-A1, WO-00/59632-A1, EP-A1-0827779, WO-99/43431-A1. Compositions that contain, inter alia, nanoscale gold particles are used as catalysts therein.

Methods for the selective separation of the partial oxidation products from the starting materials and the by-products of the above-mentioned partial oxidation are not, however, disclosed.

Methods of purifying alkene oxides, such as, for example, propene oxide, on solid activated carbons are known in principle.

U.S. Pat. No. 4,692,535 discloses, for example, the separation of high molecular weight poly(propene oxide) from propene oxide by contact with activated carbons.

U.S. Pat. Nos. 4,187,287 and 5,352,807, and EP-A1 0 736 528 disclose the separation of various organic impurities from alkene oxides, such as propene oxide and butene oxide, by treatment with solid activated carbons.

However, the selective adsorption of partial oxidation products from catalytic gas-phase direct oxidation reactions using molecular oxygen and a reducing agent is not described.

Partial oxidation using an oxygen-hydrogen mixture as in the present invention occurs in a temperature range from 140 to 210° C., which is considerably lower than the temperature at which partial oxidation wherein only oxygen, that is to say no additional hydrogen, is employed (e.g. the partial oxidation of ethene to ethene oxide; T=210-240° C.).

The low reaction temperature of <<210° C. in the process according to the present invention using oxygen and hydrogen has the result that virtually no total oxidation takes place, and therefore, only traces of carbon dioxide are formed. Instead of carbon dioxide, however, the product spectrum of the present process contains, in addition to the epoxide as the principal product, many other partial oxidation products, such as, for example, aldehydes, ketones, acids, esters, and ethers, in low concentrations. Such by-products can lower the pH value in aqueous systems, and hence reduce the stability of the epoxide.

The selective separation of partial oxidation products from non-condensable starting material gases—such as hydrocarbon, oxygen, hydrogen, diluent gas—water, water vapor and, especially, acid-reacting by-products, such as carboxylic acids and aldehydes, is not disclosed in the relevant art.

All the published Patents and Patent Applications relating to the selective oxidation of hydrocarbons in the presence of oxygen and a reducing agent achieve only a low hydrocarbon conversion of less than 10%. Accordingly, when implemented industrially, all the processes operate with very large amounts of recycling gas. The isolation of very small volumes of valuable products (e.g. 2 vol. % of hydrocarbon oxide) from large amounts of gas (e.g. 98 vol. % of gas consisting of hydrocarbon, hydrogen, oxygen, water, acetaldehyde, propionaldehyde, acetone, acetic acid, formaldehyde) is very complex. Therefore, the economy of the described selective oxidations is decisively determined by the costs of isolating the valuable product.

### SUMMARY OF THE INVENTION

The object of the present invention is to provide a process for separating the product from the reaction mixture in the preparation of hydrocarbon oxides by the partial oxidation of hydrocarbons in the presence of oxygen, at least one reducing agent, and a catalyst.

A further object of the present invention is to provide a process in which a high overall conversion of alkene to alkene oxide is achieved.

In accordance with the present invention, this is achieved by a process for the catalytic partial oxidation of hydrocarbons which comprises passing the reaction mixture through a catalyst-containing layer, wherein the reaction mixture comprises hydrocarbons, oxygen and at least one reducing agent, and adsorbing the partially oxidized hydrocarbon in a downstream adsorbent-containing layer.

### BRIEF DESCRIPTION OF THE FIGURE

The FIGURE is a flow diagram illustrating the partial oxidation of propylene to propylene oxide in accordance with the present invention.

### DETAILED DESCRIPTION

The term hydrocarbon, as used herein, is understood as meaning unsaturated or saturated hydrocarbons such as, for example, olefins or alkanes, which may also contain hetero atoms such as, for example, N, O, P, S or halogen atoms. The organic component to be oxidized may be acyclic, monocyclic, bicyclic or polycyclic, and may be monoolefinic, diolefinic or polyolefinic.

In the case of hydrocarbons having two or more double bonds, the double bonds may be conjugated or non-conjugated. The hydrocarbons from which the oxidation products are preferably formed are those hydrocarbons that yield oxidation products whose partial pressure is sufficiently low so as to enable removal of the product from the catalyst continuously. Preference is given to unsaturated and saturated hydrocarbons having from 2 to 20 carbon atoms, preferably from 2 to 12 carbon atoms, and most preferably include compounds such as ethene, ethane, propene, propane, isobutane, isobutylene, 1-butene, 2-butene, cis-2-butene, trans-2-butene, 1,3-butadiene, pentene, pentane, 1-hexene, hexenes, hexane, hexadiene, cyclohexene, benzene.

The oxygen can be used in a wide variety of forms. For example, oxygen suitable for the present invention can be present in the form of molecular oxygen, air, and/or nitrogen oxide. Molecular oxygen is preferred.

Hydrogen is a particularly suitable compound to be used as the reducing agent in the present invention. Any known hydrogen source can be used, such as, for example, pure hydrogen, cracker hydrogen, synthesis gas, or hydrogen from the dehydrogenation of hydrocarbons and alcohols. In another embodiment of the invention, the hydrogen can also be produced in situ in an upstream reactor by, for example, dehydrogenation of propane or isobutane or of alcohols such as isobutanol. The hydrogen may also be introduced into the reaction system in the form of a complex-bonded species such as, for example a catalyst-hydrogen complex.

In addition to the absolutely necessary starting material gases described above, a diluent gas may optionally also be used. Examples of suitable diluent gases include gases such as nitrogen, helium, argon, methane, carbon dioxide, carbon monoxide or similar, predominantly inert gases. It is also possible to use mixtures of the described inert components. The addition of inert components is often advantageous for transportation of the heat that is liberated in the exothermic oxidation reaction, and from the point of view of safety. If the process according to the invention is carried out in the gas phase, gaseous diluent components are preferably used, such as, for example, nitrogen, helium, argon, methane and, optionally, water vapor and carbon dioxide. Although water vapor and carbon dioxide are not totally inert, they frequently have a positive effect at low concentrations (<2 vol. %) of total reaction gas composition.

The relative molar ratios of hydrocarbon, oxygen, reducing agent (especially hydrogen) and, optionally, a diluent gas of feed or cycle gas composition can be varied within wide limits.

The range from 1 to 30 mol %, particularly preferably from 5 to 25 mol %, based on the total number of moles in the gas stream, of oxygen is preferably used.

An excess of hydrocarbon, based on oxygen used (on a molar basis), is preferably employed. The hydrocarbon content is typically greater than 1 mol % and less than 96 mol %, based on the total number of moles in the gas stream. Hydrocarbon contents in the range from 5 to 90 mol %, particularly preferably from 20 to 85 mol %, are preferably used. The molar amount of reducing agent (especially of hydrogen), relative to the total number of moles of hydrocarbon, oxygen, reducing agent and diluent gas, can be varied within wide limits. Typical reducing agent contents are greater than 0.1 mol %, preferably from 2 to 80 mol %, particularly preferably from 3 to 70 mol %.

As catalysts there are advantageously used compositions containing noble metal particles having a diameter of less than 51 nm on a support material containing titanium and/or silicon.

Gold and/or silver are preferably used as the noble metal particles. The gold particles preferably have a diameter in the range of from 0.3 to 10 nm, preferably of from 0.9 to 9 nm and particularly preferably of from 1.0 to 8 nm. The silver particles preferably have a diameter in the range of from 0.5 to 50 nm, preferably of from 0.5 to 20 nm and particularly preferably of from 0.5 to 15 nm.

There are used as catalyst support materials preferably those support materials described in, for example, DE-A1-199 59 525 and DE-A1-100 23 717, the disclosures of which are herein incorporated by reference. In particular, the catalyst support materials are preferably organic-inorganic hybrid materials (hybrid support materials, ormosils).

Organic-inorganic hybrid materials within the scope of the present invention are typically organically modified glasses, which are preferably formed in sol-gel processes by means of hydrolysis and condensation reactions of soluble precursor compounds and contain non-hydrolysable terminal and/or bridging organic groups in the network. Such materials and their preparation are disclosed, inter alia, in DE-A1-199 59 525, DE-A1-100 23 717, the disclosures of which are herein incorporated by reference.

Very particularly preferred hybrid support materials having a content of free silicon-hydrogen units incorporated and/or intercalated in the sol-gel network can be prepared particularly advantageously from titanium and silane precursor compounds as described in DE-A1-199 59 525, the disclosure of which is herein incorporated by reference.

For the generation of gold particles on the support materials there are suitable the processes disclosed in, for example, U.S. Pat. No. 5,623,090, WO-98/00413-A1, WO-98/00415-A1, WO-98/00414-A1, WO-00/59632-A1, EP-A1-0827779 and WO-99/43431-A1, the disclosures of which are herein incorporated by reference. Suitable process include processes such as, for example, deposition-precipitation, co-precipitation, impregnation in solution, incipient wetness, colloid processes, sputtering, CVD (Chemical Vapor Deposition), PVD (Physical Vapor Deposition) and microemulsion. The methods incipient wetness, solvent impregnation, and a combination of impregnation of the support materials with noble metal precursors and then immediate drying by spray or fluidised bed technology as disclosed in DE-A1-199 59 525 and DE-A1-100 23 717, the disclosures of which are herein incorporated by reference, are particularly advantageous.

The support materials may also contain amounts of promoter metals from group 5 of the periodic system according to IUPAC (1985), such as vanadium, niobium and tantalum, preferably tantalum; from group 3, preferably yttrium; from group 4, preferably zirconium; from group 8, preferably Fe; from group 15, preferably antimony; from group 13, preferably aluminium, boron, thallium; and metals of group 14, preferably germanium. The additional metals (promoters) are frequently present in the form of oxides.

The noble-metal-containing compositions according to the invention can be used at temperatures >10° C., preferably in the range from 80 to 250° C., particularly preferably in the range from 120 to 215° C. At the high temperatures, steam can be produced in coupled installations as energy carrier. With skillful process management, the steam can be used, for example, for working up the product.

The oxidation reaction is advantageously carried out at elevated reaction pressures. Reaction pressures >1 bar, particularly preferably from 2 to 50 bar, are preferred.

The catalyst load can be varied within wide limits. Catalyst loads of from 0.5 to 100 liters of the above-mentioned feed or cycle gas per ml of catalyst and per hour are preferably used, and catalyst loads of from 2 to 50 litres of gas per ml of catalyst and per hour are most preferably used.

In the catalytic oxidation of hydrocarbons in the presence of hydrogen, water is generally formed as a product coupled with the corresponding selective oxidation product.

Surprisingly, it is possible to continuously separate the partial oxidation products formed in the direct oxidation in the presence of oxygen and of a reducing agent from the reaction mixture, even in the presence of water and/or water vapor and of acid-reacting by-products, by selective adsorption on suitable adsorbents without decomposition of the adsorption products.

Therefore, suitable adsorbents include all solids that are capable of adsorbing partially oxidized hydrocarbons without decomposition, even in the presence of water and/or water vapor and of acid-reacting by-products. The adsorbent-containing layer must not initiate any consecutive reactions of the adsorbed partial oxidation products.

As adsorbents there are preferably used zeolites, or molecular sieves, and/or activated carbons. In the case of zeolites, preference is given to hydrophobic zeolites having pore sizes in the range from 0.3 to 100 nm, more preferably from 3.1 to 50 nm, such as Wesalith DAZ F20 (Degussa A G) and Wesalith DAY F20 (Degussa A G). Organically modified zeolites such as, for example, zeolites modified by silylation or treatment with fluoroorganic materials, can likewise advantageously be used.

Water is frequently adsorbed only incompletely on zeolites which are relatively hydrophobic. In various processes, such as, for example, pressure-change or temperature-change ad(de)sorption, it may therefore be advantageous to provide downstream of the adsorbent-containing layer a further layer suitable for the adsorption of water, consisting of, for example, 3A molecular sieves.

After a certain time, the adsorbed reaction product (hydrocarbon oxide) must be removed from the adsorbent again.

The adsorption and subsequent desorption can be carried out according to known processes, such as pressure-change adsorption/desorption, temperature-change adsorption/desorption, or desorption by treatment with water vapor.

The adsorption of hydrocarbon oxide is promoted by increasing pressures and/or falling temperatures, and is reduced by heating and/or a reduction in pressure.

The adsorption of hydrocarbon oxide is advantageously carried out at the reaction pressure of approximately from 1 to 30 bar. The subsequent desorption of hydrocarbon oxide then advantageously takes place at reduced pressure. For economic reasons, a compromise must be found between the ready desorption of hydrocarbon oxide at low pressures and the costs of the subsequent gas compression. A pressure difference between adsorption and desorption of <30 bar, particularly preferably of <25 bar, is preferably used.

Since the adsorption is reduced by an increase in temperature, adsorbed hydrocarbon oxide can also be desorbed by heating of the loaded adsorber bed. In order to maintain complete hydrocarbon oxide selectivity, the temperature in the adsorber during the “regeneration” process step should not exceed 200° C., preferably 180° C.

The stream of gas depleted of partial oxidation products is preferably fed back into the reactor to be reacted again, optionally after further purification such as, for example, drying. That stream of gas consists essentially of unconverted hydrocarbon, reducing agent, oxygen and, optionally, a diluent gas. As a result of this cyclic procedure with regular separation of the reaction products, substantially increased overall conversions can be achieved. The concentration of the reaction products in the adsorbent-containing layer considerably reduces the outlay in terms of working up.

A flow diagram of a process for the oxidation of propylene to propylene oxide in accordance with the present invention using two adsorbers is illustrated in the FIGURE. In the FIGURE, feed streams 1 (or cycle gas stream) (propylene (C₃H₆), hydrogen (H₂), and oxygen (O₂)) enter the reactor 2 where the oxidation occurs over the catalyst layer, the partially oxidized reaction mixture leaving the reactor 2 moves downstream to the adsorbers 3 and 4, which ad(de)sorb the propylene oxide (PO) from the reaction mixture, the propylene oxide (PO) 5 is removed as the product, non-converted gas 6 and 7 (primarily propylene (C₃H₆), hydrogen (H₂) and oxygen (O₂)) are recirculated back to the reactor 2.

In accordance with the present invention, the adsorbent-containing layers (i.e. the adsorbers) are advantageously operated in an alternating manner with each other, particularly where there are three or more adsorbers. It is preferred that one adsorber (or adsorbent-containing layer) is in the “loading” process step, the second adsorber (i.e. adsorbent-containing layer) is in the “regeneration” process step and the third adsorber (i.e. adsorbent-containing layer), where present, and also any further adsorbers present, is (are) in the “stand-by” process step. The product-containing gas stream is fed, for example, by means of a fan, over the adsorber in the “loading” position. The product-containing gas stream advantageously flows through the adsorber from bottom to top.

During the loading procedure, the mass transfer zone advantageously migrates from bottom to top in the adsorber layer. The product concentration is advantageously monitored and recorded by an analytical device (e.g. GC device). When a limit concentration is reached, adsorption can be changed over to the adsorber that is on stand-by. The previously loaded adsorber is changed over to regeneration.

The change-over from “loading” to “regeneration” takes place, for example, either automatically via an FID-GC (FID: Flame Ionisations Detector) measurement (analysis) or within particular time intervals or manually by hand.

“Regeneration” of the adsorber can take place in several ways. For example, regeneration of adsorbers can be by pressure-change ad(de)sorption, temperature-change ad(de)sorption, or by means of steam.

Regeneration of the adsorber bed by means of steam advantageously comprises the working steps of treating the adsorber bed with steam, flushing with an inert gas such as, for example, nitrogen, drying, and, optionally, cooling. The desorption of hydrocarbon oxide is preferably carried out using superheated steam at a temperature in the range from 80 to 150° C., at normal pressure or at elevated apparatus pressures. Treatment of the adsorbers with steam advantageously takes place countercurrently to the hydrocarbon oxide-containing reaction gas, from top to bottom. Such a procedure ensures that the uppermost adsorber layer remains virtually free of hydrocarbon oxide. By means of the treatment with steam, the adsorber bed is heated to a temperature in the range of, for example, from 70 to 150° C. and the adsorbed hydrocarbon oxide is desorbed. The hydrocarbon oxide is transported with the stream of steam to the base of the adsorber, where it leaves the adsorber via the regenerate pipe.

Condensation of the steam/hydrocarbon oxide mixture takes place in the regenerate coolers.

There is used as the cooling medium, for example, cooling water or brine at, for example, 20° C., countercurrently to the operating medium.

The condensed and cooled regenerate preferably flows by gravity to the sump container, from where it is conveyed by means of a pump to the hydrocarbon oxide separation.

After treatment with steam, the adsorber layer is hot and moist. In the case of drying and cooling by means of air, there is the risk that explosive gas mixtures will form. The maximum oxygen content in the system should therefore be <20 vol. % of total feed or cycle gas composition. The oxygen content is monitored by, for example, an oxygen-measuring device.

Drying and cooling of the adsorber layer is therefore advantageously carried out by means of inert gases, such as, for example, nitrogen. For economic reasons, flushing with inert gas takes place in a closed circuit.

To that end, the adsorber is first flushed with nitrogen (approximately three times the volume of the adsorber). The nitrogen displaces the steam and at the same time strips residual amounts of hydrocarbon oxide from the contact water adhering to the absorber.

Drying of the adsorber layer preferably takes place in a closed pipe system by means of inert gas (e.g. nitrogen). The stream of nitrogen gas, for example, is conveyed over the adsorber bed from bottom to top by means of a fan. The stream of nitrogen is heated in the steam-heated heat exchanger to a temperature in the range, for example, from 100 to 130° C. and heats the adsorber layer, which was initially cooled to the cooling limit temperature by evaporation of water, and thereby desorbs adsorbed water. After leaving the adsorber, the warm, moist stream of nitrogen is condensed and cooled by means of, for example, two series connected heat exchangers. The water/solvent condensate that forms is passed into the sump container. As soon as the temperature in the middle of the adsorber bed rises, the drying operation is terminated.

The cooling operation takes place analogously to the drying operation, except that the heater is switched off. At the beginning of the cooling, the lower layer of the adsorber bed is cooled, while the upper layer is still heated and dried by the heated stream of nitrogen displaced during cooling. At the same time, further solvents are desorbed from the upper adsorber layer.

As soon as the adsorber has cooled to, for example, 30° C., the fully regenerated adsorber can be loaded again.

The process according to the invention is used particularly preferably for oxidizing propene (propylene) to propene (propylene) oxide.

The process according to the invention is also suitable for the oxidation of hydrocarbons in the liquid phase. When the invention is implemented in the liquid phase, an inert liquid that is stable to oxidation and to heat is preferably chosen, such as an alcohol, a polyalcohol, a polyether, a halogenated hydrocarbon, a silicone oil. Olefins, for example, are selectively converted in the liquid phase to epoxides using the described catalysts both in the presence of organic hydroperoxides (ROOH) and in the presence of hydrogen peroxide or in the presence of oxygen and hydrogen.

The characteristic properties of the present invention are illustrated in the following Examples by means of test reactions. The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

Specification for Testing the Adsorption of Catalytically Prepared Crude Propene Oxide on Suitable Solid Adsorbers (Test Specification):

A tubular metal reactor having an inside diameter of 10 mm and a length of 30 cm was used, the temperature of which was controlled by means of an oil thermostat. The reactor was supplied with starting material gases by means of a set of four mass-flow regulators (hydrocarbon, oxygen, hydrogen, nitrogen). For the reaction, 1 g of catalyst (2×2 mm; see catalyst preparation specification below) was introduced at 160° C. and 3 bar. The standard catalyst load was 5 liters of gas/(g cat.×h). Propene was used as the hydrocarbon. The catalyst productivity when propene was used as the hydrocarbon was 200 g of propene oxide/(kg cat.×h). The reaction gas stream was subsequently passed under system pressure through a downstream adsorber (metal tube, 10 mm inside diameter and 30 cm length; filled with adsorber fixed bed). For the purposes of desorption, the loaded adsorber was reduced to a pressure of 100 mbar and the desorbed material was collected by means of a cold trap (−40° C.).

For carrying out the oxidation reactions, a gas stream, hereinafter always referred to as the standard gas composition, was chosen: it comprised C₃H₆/H₂/O₂: 60/30/10% vol. %. The reaction gases were analyzed quantitatively by gas chromatography upstream and downstream of the adsorber. Separation of the individual reaction products by gas chromatography was carried out by a combined FID/WLD method, in which three capillary columns are passed through:

FID: HP-Innowax, inside diameter 0.32 mm, length 60 m, layer thickness 0.25 μm.

WLD: Series connection of

HP-Plot Q, inside diameter 0.32 mm, length 30 m, layer thickness 20 μm

HP Plot 5 A molecular sieve, inside diameter 0.32 mm, length 30 m, layer thickness 12 μm.

(FID is flame ionization detector; WLD is heat conductivity detector)

Catalyst Preparation:

This Example first describes the preparation of a powdered catalytically active organic-inorganic hybrid material consisting of a silicon- and titanium-containing, organic-inorganic hybrid material having free silane hydrogen units, which has been provided with gold particles (0.04 wt. %) by means of incipient wetness. The finely powdered catalyst material was then converted into extrudates.

184.29 g of methyltrimethoxysilane (1.35 mol) and 25.24 g of triethoxysilane (153.6 mmol) were placed in a vessel. 44.79 g of p-toluenesulfonic acid (0.1 n) were added, and 17.14 g of tetrapropoxytitanium, dissolved in 40 g of ethanol, were then added thereto. After an ageing time of 12 hours, the gel was washed twice using 200 ml of hexane each time, and dried for 2 hours at RT and for 8 hours at 120° C. in air.

10.1 g of dried sol-gel material was impregnated with 5 g of a 0.16 % solution of HAuCl₄×H₂O in methanol, with stirring (incipient wetness), dried at RT in a stream of air, and then tempered for 8 hours at 120° C. in air and then for 5 hours at 400° C. under a nitrogen atmosphere. The catalytically active organic-inorganic hybrid material so prepared contained 0.04 wt. % gold.

Extrudate Formation:

8.5 g of organic-inorganic hybrid material, synthesised according to the above catalyst preparation, were mixed intensively for 2 hours with 5 g of silicon dioxide sol (Levasil, Bayer, 300 m²/g, 30 wt. % SiO₂ in water) and 1.0 g of SiO₂ powder (Ultrasil VN3, Degussa). 2 g of sodium silicate solution (Aldrich) were added to the resulting plastic mass, and the mixture was homogenized intensively for 5 minutes and then formed into 2 mm extrudates in an extruding machine. The extrudates so produced were dried first for 8 hours at room temperature and then for 5 hours at 120° C., and were then tempered for 4 hours under a nitrogen atmosphere at 400° C. The mechanically stable molded body has high resistance to lateral pressure.

The tempered 2×2 mm molded bodies were used as catalyst in the gas-phase epoxidation of propene using molecular oxygen in the presence of hydrogen.

### Example 1

The total reaction gas composition at reactor outlet (analysis at the reactor outlet; upstream of the adsorber) contained 1.5 vol. % propene oxide, 2.5 vol. % water, 0.15 vol. % by-products (inter alia acetaldehyde, propionaldehyde, acetone, acetic acid). The reaction gas or mixture was passed through an adsorber filled with 5 g of DAY F20 (Degussa). The propene oxide gas concentration downstream of the adsorber was measured by GC in dependence on time.

The propene oxide capacity of DAY F20 was approximately 200 g of propene oxide/(kg DAY×h).

| | Propene oxide concentration in the gas phase [vol. %] |
|---|---|
| Time [h] | (downstream of the adsorber) |
| 0 | 0 |
| 1 | 0 |
| 2 | 0 |
| 2.5 | 0.1 |
| 2.7 | 0.2 |
| 3 | 0.3 |
| 4 | 1.0 |
| 5 | 1.45 |
| 6 | 1.46 |

The adsorbed epoxide can be desorbed to the extent of 90% by lowering the pressure to 100 mbar. Five cycles of “loading of the adsorbent” and “regeneration of the adsorbent” were carried out. From cycle 2, the PO desorption rate was >97%.

### Example 2

The reaction gas or mixture (analysis at the reactor outlet; upstream of the adsorber) contained 1.5 vol. % propene oxide, 2.5 vol. % water, 0.15 vol. % by-products (inter alia acetaldehyde, propionaldehyde, acetone, acetic acid). The reaction gas or mixture was passed through an adsorber filled with 5 g of DAZ F20 (Degussa). The propene oxide gas concentration downstream of the adsorber was measured by GC in dependence on time.

The propene oxide capacity of DAZ F20 was approximately 100 g of propene oxide/(kg DAZ×h).

| | Propene oxide concentration in the gas phase [vol. %] |
|---|---|
| Time [h] | downstream of the adsorber |
| 0 | 0 |
| 1 | 0 |
| 2 | 0.4 |
| 2.5 | 1.4 |
| 3 | 1.46 |

The adsorbed epoxide can be desorbed to the extent of 84% by lowering the pressure to 100 mbar. Five cycles of “loading of the adsorbent” and “regeneration of the adsorbent” were carried out. From cycle 2, the PO desorption rate was >95%.

### Example 3

The reaction gas or mixture (analysis at the reactor outlet; upstream of the adsorber) contained 1.5 vol. % propene oxide, 2.5 vol. % water, 0.15 vol. % secondary products (inter alia acetaldehyde, propionaldehyde, acetone, acetic acid). The reaction gas was passed through an adsorber filled with 5 g of activated carbon (Degussa). The propene oxide gas concentration downstream of the adsorber was measured by GC in dependence on time.

The propene oxide capacity of activated carbon was approximately 200 g of propene oxide/(kg activated carbon×h).

| | Propene oxide concentration in the gas phase [vol. %] |
|---|---|
| Time [h] | downstream of the adsorber |
| 0 | 0 |
| 1 | 0 |
| 2 | 0 |
| 2.5 | 0 |
| 3 | 0 |
| 4 | 0.5 |
| 5 | 1.4 |
| 6 | 1.45 |
| 7 | 1.45 |

The adsorbed epoxide can be desorbed to the extent of 95% by lowering the pressure to 100 mbar. Five cycles of “loading of the adsorbent” and “regeneration of the adsorbent” were carried out. From cycle 2, the PO desorption rate was >95%.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the catalytic partial oxidation of hydrocarbons, comprising
(a) passing the reaction mixture through a catalyst-containing layer, wherein the reaction mixture comprises (1) one or more hydrocarbons, (2) oxygen and (3) at least one reducing agent; and
(b) adsorbing the partially oxidized hydrocarbon in a downstream adsorbent-containing layer.

2. The process of claim 1, additionally comprising:
(c) feeding the reaction gas back into the reaction in step (a) after the partially oxidized hydrocarbon is adsorbed.

3. The process of claim 1, wherein (b) the adsorption of the partially oxidized hydrocarbon is carried out in the presence of non-condensable gases.

4. The process of claim 1, wherein the adsorbent of the adsorbent-containing layer comprises zeolites and/or activated carbons.

5. The process of claim 4, wherein the adsorbents comprise hydrophobic zeolites and/or zeolites modified organically by silylation and/or by treatment with fluoroorganic compounds.

6. The process of claim 1, wherein the adsorbent, after the adsorption of the partially oxidised hydrocarbon, is regenerated by means of desorption.

7. The process of claim 6, wherein desorption is carried out by means of pressure-change desorption, temperature-change desorption or by steam treatment.
